(19) United States
(12) Patent Application Publication
(10) Pub. No.: US 2008/0226631 A1
(43) Pub. Date: Sep. 18, 2008

(54) METHODS OF MODULATING CYTOKINE ACTIVITY; RELATED REAGENTS

(75) Inventors: Gabriele Grunig, Syosset, NY (US); Rene de Waal Malefyt, Sunnyvale, CA (US)

Correspondence Address: DNAX RESEARCH INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 12/119,261

(22) Filed: May 12, 2008

Related U.S. Application Data
(63) Continuation of application No. 11/001,443, filed on Dec. 1, 2004.
(60) Provisional application No. 60/526,558, filed on Dec. 3, 2003.

(51) Int. Cl. A61K 39/395; C12Q 1/68; C12N 5/06; A61K 31/7088; A61K 49/00; A61P 3/10; A61P 11/00

(52) U.S. Cl. 424/133.1; 435/7.8; 435/6; 435/375; 424/158.1; 424/145.1; 514/44; 800/3

(57) ABSTRACT

Provided are methods of modulating cytokine activity, e.g., for the purpose of treating inflammation of the airways and lung. Also provided are reagents for use in screening for agonists or antagonists of IL-19 or IL-24.

This filing is a Continuation of co-pending application Ser. No. 11/001,443, filed Dec. 1, 2004, which claims benefit of U.S. Provisional Patent Application No. 60/526,558, filed Dec. 3, 2003.

### FIELD OF THE INVENTION

The present invention relates generally to uses of mammalian cytokines. More specifically, the invention discloses cytokine function in airway hyperreactivity.

### BACKGROUND OF THE INVENTION

The immune system functions to protect individuals from infective agents, e.g., bacteria, multi-cellular organisms, and viruses, as well as from cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. The immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Cytokines have been implicated in the pathology of a number of disorders involving airway hyperreactivity and alveolar macrophages, i.e., infiltration by or activation of alveolar macrophages (see, e.g., Abbas, et al. (eds.) (2000) Cellular and Molecular Immunology, W.B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med. 345:340-350; Riffo-Vasquez and Spina (2002) Pharmacol. Therapeutics 94:185-121; Evans, et al. (2003) Int. Rev. Immunol. 22:173-194; Lysaght, et al. (2003) Curr. Opin. Investig. Drugs 4:716-721; Engleman (2003) Semin. Oncol. 30(3 Suppl. 8):23-29).

Airway hyperreactivity, also known as airway hyperresponsiveness, which involves inappropriate airway narrowing in response to a stimulus, is a characteristic of various disorders of the airways, e.g., asthma, allergic rhinitis, bronchitis, bronchiolitis, and possibly chronic obstructive pulmonary disorder (COPD). Hyperreactivity can be triggered by, e.g., respiratory infections, smoke, and respiratory allergens. Asthma, a chronic disorder that can be fatal, affects about one in seven children in the United States, and accounts for over 15% of pediatric emergencies. The symptoms involve shortness of breath, and mucus hypersecretion (see, e.g., Crain, et al. (1995) Arch. Pediatr. Adolesc. Med. 149:893-901; Grunig, et al. (1998) Science 282:2261-2263; Crystal, et al. (eds.) (1997) The Lung, Vols. 1-2, 2^{nd} ed., Lippincott-Raven, Phila, Pa.; Holgate, et al. (2001) Allergy, 2^{nd} ed., Mosby, N.Y.; Marone (1998) Immunol. Today 19:5-9; Barnes and Lemanske (2001) New Engl. J. Med. 344:350-362).

Another airway hyperreactivity disorder is allergic rhinitis, one of the most common of all chronic conditions, involving inflammation of the upper respiratory tract, and responsible for about 2 million lost days of work every year in the United States (see, e.g., Marone (1998) Immunol. Today 19:5-9; Kumar (2001) Pharmacol. Therapeutics 91:93-104; Homer (1997) New. Engl. J. Med. 337:1461-1463; Platts-Mills and Carter (1997) New Engl. J. Med. 336:1382-1384; James (2003) Pediatrics 111:1625-1630; Robinson, et al. (1996) Pediatr. Pulmonol. 22:248-254; Beckett (2000) New Engl. J. Med. 342:406-413; Siroux, et al. (2003) Clin. Exp. Allergy 33:746-751; Kessler, et al. (2001) Annals Allergy Asthma Immunol. 87:289-295).

Airway hyperreactivity is characterized by infiltration by T cells, eosinophils, mast cells, neutrophils, and antigen presenting cells (APCs), in the airways. The APCs of the lung include DCs, B cells, and alveolar macrophages, each of which can express cytokines and contribute to airway hyperreactivity (see, e.g., Lawrence, et al. (1998) J. Pharm. Exp. Thera. 284:222-227; Alexis, et al. (2001) Am. J. Physiol. Lung Cell Mol. Physiol. 280:L369-L375; Akabari, et al. (2002) Nature Medicine 8:1024-1032; MacLean, et al. (1999) Am. J. Respir. Cell Mol. Biol. 20:379-387; Hamelmann, et al. (1999) Am. J. Respir. Cell Mol. Biol. 21:480-489; Gonzales, et al. (2000) Annals Internal Medicine 133:981-991; Li, et al. (2002) Pulmonary Pharmacol. Therapeutics 15:409-416; Zimmermann, t al. (2003) J. Allergy Clin. Immunol. 111:227-242; Riffo-Vasquez and Spina (2002) Pharmacol. Therapeutics 94:185-211). Airway macrophages, which include alveolar macrophages, dendritic cells, and pleural, interstitial, and intravascular macrophages, mediate immune responses by direct interaction with T cells and by producing cytokines, e.g., in response to pathogens and allergens (and in disorders such as asthma and allergies). Alveolar macrophages are phagocytic against bacteria, spores, fungi such as Pneumocystis carinii, Cryptococcus neoformans, viruses, e.g., respiratory syncytial virus (RSV), tumors, and cigarette smoke. Moreover, these immune cells modulate the acquired immune response by secreting cytokines (see, e.g., McNamara, et al. (2002) Brit. Med. Bull. 61:13-28; Openshaw (2002) Respir. Res. 3 (Suppl.1) S15-S20; Gordon and Read (2002) Brit. Medical Bulletin 61:45-61; Guidi-Rontani (2002) Trends Microbiol. 10:405-409; Eifuku, et al. (2000) Jpn. J. Clin. Oncol. 30:295-300; Fathi, et al. (2001) Exp. Mol. Pathol. 70:77-82; Ieong, et al. (2000) Am. J. Resp. Crit. Care Med. 162:966-970; Yi (2002) Crit. Rev. Clin. Lab Sci. 39:581-629; Friedman, et al. (1998) Semin. Respir. Infect. 13:100-108; Brummer (1998-1999) Mycopathologia 143:121-125; Vassallo, et al. (2000) Sarcoidosis Vasc. Diffuse Lung Dis. 17:130-139; Benten, et al. (2003) J. Medical Virol. 71:290-297; Rigden, et al. (2002) Immunology 106:537-548).

Alveolar macrophages have also been implicated in the pathology of chronic obstructive pulmonary disorder (COPD), a disorder involving bronchiolar infiltration with macrophages, neutrophils, and T cells, e.g., CD8⁺ T cells. COPD, the fourth leading cause of death in North America, is characterized by thickening of airway smooth muscle and inflammation of the airways. This response appears to be due to the infiltration of monocytes, macrophages, CD4⁺ T cells, CD8⁺ T cells, and neutrophils to the lungs. Alveolar macrophages, elevated in COPD, express cytokines that, in turn, promote inflammation and increase in immune cell activation. COPD involves chronic bronchitis and emphysema. Emphysema is characterized by permanent destruction of the parenchyma, airspaces distal to the terminal bronchioli, see, e.g., Hautamaki, et al. (1997) Science 277:2002-2004; Barnes (2000) Chest 117:10S-14S; Barnes (2003) Annu. Rev. Med. 54:113-129; Jeffery (1998) Thorax 53:129-136; Barnes (2000) New Engl. J. Med. 343:269-280.

Asthma and COPD, as well as alveolar macrophage-mediated defenses against infections and cancers, remain poorly understood and new therapies are needed. The present invention fulfills this need by identifying two cytokines associated with inflammation, airway hyperreactivity, and macrophage activity, and by providing reagents and methods for treatment and diagnosis of these respiratory disorders.

### SUMMARY OF THE INVENTION

The present invention is based, in part, upon the discovery that animals deficient in IL-19 or IL-24 display reduced airway hyperreactivity.

The invention provides a method of modulating an activity of a cell comprising contacting the cell with an agonist or antagonist of IL-19 (SEQ ID NOs:1 or 2) or of IL-24 (SEQ ID NOs:3 or 4); wherein the cell modulates airway hyperreactivity or airway inflammation.

Also provided is the above method wherein the cell is a monocyte or macrophage, a T cell or B cell, a dendritic cell, or an epithelial or endothelial cell; the above method wherein the macrophage is an alveolar macrophage; the above method wherein the agonist or antagonist is a binding composition derived from an antigen binding site of an antibody; and the above method wherein the agonist or antagonist specifically binds to a polypeptide or nucleic acid of IL-19 (SEQ ID NOs:1 or 2); IL-24 (SEQ ID NOs:3 or 4); IL20R1; IL-20R2; or IL-22R.

In another embodiment, the invention provides the above method of modulating an activity, wherein the agonist or antagonist comprises a polyclonal antibody; a monoclonal antibody; a humanized antibody; an Fab, Fv, or F(ab′)₂ fragment; a peptide mimetic of an antibody; a nucleic acid; or a detectable label.

In another aspect, the invention provides a method of treating a subject suffering from an airway hyperreactivity disorder or an airway inflammatory disorder comprising administering an effective amount of an agonist or antagonist of IL-19 (SEQ ID NOs:1 or 2) or of IL-24 (SEQ ID NOs:3 or 4); the above method wherein the disorder is mediated by monocytes or macrophages, T cells or B cells, dendritic cells, or epithelial or endothelial cells; and the above method wherein the macrophages are alveolar macrophages; as well as the above method wherein the disorder comprises asthma, allergic rhinitis, bronchitis; bronchiolitis, or chronic obstructive pulmonary disorder (COPD).

Yet another embodiment of the present invention provides the above method of treating a subject, wherein the antagonist reduces or inhibits airway hyperreactivity; wherein the agonist or antagonist is a binding composition derived from an antigen binding site of an antibody; wherein the agonist or antagonist specifically binds to a polypeptide or nucleic acid comprising IL-19 (SEQ ID NO:1 or 2); IL-24 (SEQ ID NO:3 or 4); IL-20R1; IL-20R2; or IL-22R; or the above method wherein the agonist or antagonist comprises a polyclonal antibody; a monoclonal antibody; a humanized antibody; an Fab, Fv, or F(ab′)₂ fragment; a peptide mimetic of an antibody; a nucleic acid; or a detectable label.

The present invention also provides a method of diagnosing an airway hyperreactivity disorder or an airway inflammatory disorder comprising contacting a sample from a test subject with a binding composition that specifically binds to a polypeptide or nucleic acid of IL-19 (SEQ ID NO:1 or 2); IL-24 (SEQ ID NO:3 or 4); IL-20R1; IL-20R2; or IL-22R; as well as the above method further comprising contacting the binding composition to a sample derived from a control subject or control sample; and comparing the binding found with the test subject with the binding found with the control subject or control sample.

Another embodiment of the present invention provides a method of screening for a compound that modulates a physiological activity, comprising contacting a candidate compound to an IL-19 knockout (IL-19KO) mouse to provide a contacted IL-19KO mouse, and determining the physiological activity in the contacted IL-19KO mouse; determining the physiological activity in an IL-19KO mouse not contacted with the candidate compound; and comparing the physiological activities of the contacted IL-19KO mouse and the non-contacted IL-19KO mouse; as well as the above method wherein the physiological activity comprises an immune activity; inflammation of the airway; airway hyperreactivity; or a proliferative activity.

Yet another embodiment of the present invention is a method of screening for a compound that modulates a physiological activity, comprising contacting a candidate compound to an IL-24 knockout (IL-24KO) mouse to provide a contacted IL-24KO mouse, and determining the physiological activity in the contacted IL-24KO mouse; determining the physiological activity in an IL-24KO mouse not contacted with the candidate compound; and comparing the physiological activities of the contacted IL-24KO mouse and the non-contacted IL-24KO mouse; as well as the above method wherein the physiological activity comprises an immune activity; inflammation of the airway; airway hyperreactivity; or a proliferative activity.

An additional aspect of the present invention is a method for treating or diagnosing obesity or diabetes comprising administering an effective amount of an agonist
or antagonist of IL-19; or of IL-24.

### DETAILED DESCRIPTION OF THE PREFERRED EMBODIMENTS

As used herein, including the appended claims, the singular forms of words such as “a,” “an,” and “the,” include their corresponding plural references unless the context clearly dictates otherwise.

All references cited herein are incorporated herein by reference to the same extent as if each individual publication or patent application was specifically and individually indicated to be incorporated by reference.

### I. Definitions.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compositions derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to treatment of a human subject, research subject, veterinary subject, animal, or cell, refers to contact of a pharmaceutical, therapeutic, diagnostic agent or composition, or placebo, to the human subject, animal, or cell. Treatment of a cell encompasses contact of a reagent to the cell, as well as contact of a reagent to a fluid, where the fluid is in contact with the cell. “Administration” and “treatment” also encompass ex vivo treatment, e.g., ex vivo treatment to a cell, tissue, or organ, followed by contact of the cell, tissue, or organ, to the subject or animal, even where the agent or composition has been metabolized, altered, degraded, or removed, during or after the ex vivo treatment.

“Candidate compound” refers, e.g., to a molecule, complex of molecules, or mixture of molecules, where the candidate compound is used in the development or identification of a therapeutic or diagnostic agent. Testing or screening of a candidate compound is used to determine if the compound will be useful as a therapeutic or diagnostic. “Candidate compounds” encompass, e.g., polypeptides, antibodies, natural products, synthetic chemicals, organic compounds, inorganic compounds, and combinations thereof with a second therapeutic or diagnostic, or a carrier, diluent, stabilizer, or excipient.

“Disorder” refers to a pathological state, or a condition that is correlated with or predisposes to a pathological state. “Infectious disorder” refers, e.g., to a disorder resulting from a microbe, bacterium, parasite, virus, and the like, as well as to an inappropriate, ineffective, or pathological immune response to the disorder. “Oncogenic disorder” encompasses a cancer, transformed cell, tumor, displasia, angiogenesis, metastasis, and the like, as well as to an inappropriate, ineffective, or pathological immune response to the disorder.

A “gene” encompasses the coding region of a polypeptide and any regulatory sequences, e.g., promoters, operators, enhancers, and transcriptional start and stop signals. The coding region may comprise one, continuous open reading frame (ORF), or it may comprise more than one ORF, i.e., it may be interrupted by one or more introns.

“Effective amount” means, e.g., an amount of an IL-19 agonist, antagonist, or binding compound or composition, or an IL-24 agonist, antagonist, or binding compound or composition, sufficient to ameliorate a symptom or sign of a disorder, condition, or pathological state. “Effective amount” also relates to an amount of an IL-19 agonist, antagonist, or binding compound or composition, or an IL-24 agonist, antagonist, or binding compound or composition, sufficient to diagnose a symptom or sign of a disorder, condition, or pathological state.

“Expression” refers to a measure of mRNA or polypeptide encoded by a specific gene. Units of expression may be a measure of, e.g., the number of molecules of mRNA or polypeptide/mg protein in a cell or tissue, or in a cell extract or tissue extract. The units of expression may be relative, e.g., a comparison of signal from control and experimental mammals or a comparison of signals with a reagent that is specific for the mRNA or polypeptide versus with a reagent that is non-specific.

“Inflammatory disorder” means a disorder or pathological condition where the pathology results, in whole or in part, from an increase in the number and/or increase in activation of cells of the immune system, e.g., of T cells, B cells, monocytes or macrophages, alveolar macrophages, dendritic cells, NK cells, NKT cells, neutrophils, eosinophils, or mast cells.

“Knockout” (KO) refers to the partial or complete reduction of expression of at least a portion of a polypeptide encoded by a gene, e.g., IL-19 or IL-24, where the gene is endogenous to a single cell, selected cells, or all of the cells of a mammal. KO also encompasses embodiments where biological function is reduced, but where expression is not necessarily reduced, e.g., an IL-19KO polypeptide comprising an expressed IL-19 polypeptide that contains an inserted inactivating peptide, oligopeptide, or polypeptide. Disruptions in a coding sequence or a regulatory sequence are encompassed by the knockout technique. The cell or mammal may be a “heterozygous knockout”, where one allele of the endogenous gene has been disrupted. Alternatively, the cell or mammal may be a “homozygous knockout” where both alleles of the endogenous gene have been disrupted. “Homozygous knockout” is not intended to limit the disruption of both alleles to identical techniques or to identical outcomes at the genome. Included within the scope of this invention is a mammal in which one or both IL-19 alleles and/or one or both IL-24 alleles have been knocked out. “Transgenic” refers to a genetic change, produced by a technique of genetic engineering, that is stably inherited. Transgenic methods, cells, and animals, includes genetic changes that result from use of a knockout technique.

A “marker” relates to the phenotype of a cell, tissue, organ, animal, e.g., of an IL-19KO mouse or IL-24KO mouse, or human subject. Markers are used to detect cells, e.g., during cell purification, quantitation, migration, activation, maturation, or development, and may be used for both in vitro and in vivo studies. An activation marker is a marker that is associated with cell activation.

“Sensitivity,” e.g., sensitivity of receptor to a ligand, means that binding of a ligand to the receptor results in a detectable change in the receptor, or in events or molecules specifically associated with the receptor, e.g., conformational change, phosphorylation, nature or quantity of proteins associated with the receptor, or change in genetic expression mediated by or associated with the receptor.

“Soluble receptor” refers to receptors that are water-soluble and occur, e.g., in extracellular fluids, intracellular fluids, or weakly associated with a membrane. Soluble receptor further refers to receptors that are engineered to be water soluble.

“Specificity of binding,” “selectivity of binding,” and the like, refer to a binding interaction between a predetermined ligand and a predetermined receptor that enables one to distinguish between the predetermined ligand and other ligands, or between the predetermined receptor and other receptors. “Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, binds to its antigen with an affinity that is at least two fold greater, preferably at least ten times greater, more preferably at least 20-times greater, and most preferably at least 100-times greater than the affinity to any other antigen. In a preferred embodiment the antibody will have an affinity that is greater than about 10⁹ liters/mol, see, e.g., Munsen, et al. (1980) Analyt. Biochem. 107:220-239.

### II. General.

IL-19 and IL-24 (a.k.a. mda-7) are members of the IL-10 family of cytokines, sharing 21-22% amino acid sequence identity with IL-10. IL-10, IL-19, and IL-24 reside on human chromosome 1q32, and are expressed, e.g., by T cells. Rodent orthologues of IL-24 have been identified, e.g., c49a (a.k.a. mob-5) of the rat and FISP of the mouse, where these orthologues function somewhat differently than human IL-24. Receptors of IL-19 and IL-24 have also been identified. The IL-19 receptor complex is a heterodimer of IL-20R1 (a.k.a. crf2-8) and IL-20R2 (a.k.a. DIRS1; crf2-11). IL-24 binds to two different receptor complexes, i.e., the heterodimer of IL-22R1 and IL-20R2 and the heterodimer of IL-20R1 (a.k.a. IL-20A) and IL-20R2 (a.k.a. IL-20RB) (see, e.g., Gallagher, et al. (2000) Genes Immunity 1:442-450; Dumoutier, et al. (2001) J. Immunol. 167:3545-3549; Fickenscher, et al. (2002) Trends Immunol. 23:89-96; Parrish-Novak, et al. (2002) J. Biol. Chem. 277:47517-47523; Wang, et al. (2002) J. Biol. Chem. 277:7341-7347; U.S. Pat. Pub. No. US 2003/0078381).

A number of factors can stimulate IL-19 or IL-24 expression which, in turn, can provoke events such as apoptosis, or expression of other cytokines. Stimulatory factors include lipopolysaccharide (LPS), IL-4, granulocyte monocyte colony stimulating factor (GM-CSF), and phytohemagluttinin (PHA).

IL-19 and IL-24 and/or their receptor complexes have been identified with a number of physiological activities. IL-19 receptor expression has been associated with psoriasis. IL-24 inhibits angiogenesis as well as the differentiation and migration of endothelial cells. Additionally, IL-24 provokes expression of cytokines, e.g., interleukin-6 (IL-6) and tumor necrosis factor alpha (TNFalpha). C49a, the rat orthologue of IL-24, is associated with increased cell proliferation, e.g., wound healing and ras oncogene transformation (see, e.g., Ramesh, et al. (2003) Cancer Res. 63:5105-5113; Sauane, et al. (2003) Cytokine Growth Factor Revs. 14:35-51; Parrish-Novak, et al., supra; Wolk, et al. (2002) J. Immunol. 168:5397-5402; Liao, et al. (2002) J. Immunol. 169:4288-4297; Ghoreschi, et al. (2003) Nature Medicine 9:40-46; Caudell, et al. (2002) J. Immunol. 168:6041-6046; Dumoutier, et al., supra; Sarkar, et al. (2002) Proc. Natl. Acad. Sci. USA 99:10054-10059; Jiang, et al. (1996) Proc. Natl. Acad. Sci. 93:9160-9165; Chang, et al. (2003) J. Biol. Chem. 278:3308-3313; Fickenscher, et al. (2002) Trends Immunol. 23:89-96; Gallagher, et al. (2000) Genes Immunol. 1:442-450; Vandenbroeck, et al. (2002) J. Biol. Chem. 277:25668-25676; Sauane, et al. (2003) J. Cellular Physiol. 196:334-345).

The biological effects of IL-19 and IL-24, and agonists and antagonists thereto, can be determined with the use of certain phenotypic “markers” associated with a cell. CD11c⁺ MHC low cells were found to increase in the IL-24KO mouse, as compared to the wild type control mouse, as determined by analysis of cells in the bronchoalveolar lavage fluid BAL. CD11c, a marker of immune cell development and cell activation, is a member of the CD11 family of α-integrin proteins. CD11 mediates cell adhesion and migration, including movement of immune cells across epithelial cells to the airway lumen. CD11c is a marker for activation of, e.g., DCs, macrophages, T cells, and B cells, see, e.g., Kadowaki, et al. (2001) J. Immunol. 166:2291-2295; Kadowaki, et al. (2001) J. Exp. Med. 194:863-869; Liu (2002) Human Immunology 63:1067-1071; Shelley, et al. (2002) J. Immunol. 168:3887-3893; Kidney and Proud (2000) Am. J. Respir. Cell Mol. Biol. 23:389-395; Taborda and Casadevall (2002) Immunity 16:791-802.

MHC Class II expression increases in the IL-24KO mouse, as compared to the wild type control mouse, as determined with measurements of cells in the BAL. MHC Class II is a marker for cell activation or maturation, e.g., of DCs, monocytes, macrophages, B cells, T cells, and endothelial cells. Once an antigen presenting cell (APC), such as a DC or B cell, expresses MHC Class II, the APC can activate other types of cells, i.e., T cells, see, e.g., Waldburger, et al. (2001) J. Exp. Med. 194:393-406; Pai, et al. (2002) J. Immunol. 169:1326-1333; Villadangos, et al. (2001) Immunity 14:739-749; Xaus, et al. (2000) J. Immunol. 165:6364-6371; Kwak, et al. (2000) Nature Medicine 6:1399-1402; Mach, et al. (1996) Annu. Rev. Immunol. 14:301-331.

The proportion of dendritic cells expressing B220 was greater in non-challenged IL-19KO mice than in non-challenged wild type control mice. B220 is a protein found, e.g., on DCs. This protein has been associated with greater or lesser DC activity, depending on the context. CD11c⁺B220⁺ DCs are attracted to more types of chemokines than CD11C⁺B220⁻ DCs though, in apparent contrast, CD11c⁺B220⁺ DCs have a lesser ability to stimulate T cell proliferation than CD11c⁺B220⁻ DCs (Brawand, et al. (2002) J. Immunol. 169:6711-6719).

### III. Genetic Alteration of Nucleic Acids, Cells, and Organisms.

Nucleic acids, cells, and organisms can be genetically modified, e.g., by altering or deleting existing nucleic acid sequences, or by introducing new chromosomal or extra-chromosomal sequences. Genetic alteration encompasses introducing new genes, mutating or knocking out existing genes, and altering the regulatory properties of genes. These alterations include covalent modifications as well as non-covalent modification of the chromosome or of extra-chromosomal elements.

A cytokine knockout (KO) construct, e.g., an IL-19KO construct or IL-24KO construct, is typically prepared by isolating a portion of the genomic or cDNA cytokine nucleotide sequence and inserting a marker sequence into the cytokine sequence. The cytokine DNA molecule will be long enough to provide sufficient complementary sequence for recognition with chromosomal DNA, i.e., homologous recombination, when the KO construct is introduced into the genomic DNA of an embryonic stem (ES) cell.

A naturally occurring genomic cytokine fragment or cDNA molecule, i.e., encoding IL-19 or IL-24, to be used in preparing the KO construct can be obtained using methods well known in the art, e.g., polymerase chain reaction (PCR) amplification of a particular DNA sequence, or screening a genomic library prepared from cells or tissues that contain the cytokine gene, where screening uses a cDNA probe encoding at least a portion of the same or a highly homologous cytokine gene in order to obtain at least a portion of the cytokine genomic sequence. Alternatively, if a cDNA sequence is to be used in a KO construct, the cDNA may be obtained by screening a cDNA library.

The proper position for marker gene insertion is one that will serve to decrease or prevent transcription or translation of the full length endogenous cytokine gene, or one that allows translation but where the cytokine protein is biologically inactive. The insertion procedure can be accomplished with or without deletion of sequence from the cytokine gene, i.e., with or without deletion of introns and/or exons.

A marker gene is usually operably linked to its own promoter or to another strong promoter, e.g., the thymidine kinase (TK) promoter or the phosphoglycerol kinase (PGK). The marker gene need not have its own promoter attached, as it may be transcribed using the promoter of the gene to be knocked out. Preferred marker genes are any antibiotic resistance gene such as neo, which encodes a neomycin resistance gene, or beta-gal, which encodes beta-galactosidase. In some cases, it will be preferable to insert the marker sequence in the reverse or antisense orientation with respect to the cytokine nucleic acid sequence. Reverse insertion is preferred where the marker gene is operably linked to a particularly strong promoter. The ligated DNA KO construct may be transfected directly into ES cells or it may first be placed into a suitable vector for amplification prior to insertion.

The IL-19KO or IL-24KO construct is typically transfected into an ES, where the ES cell line used is selected for its ability to integrate into and become part of the germ line of a developing embryo so as to create germ line transmission of the KO construct. ES cell lines for generating KO mice include, e.g., murine cell lines D3 and E14 (American Type Culture Collection, Rockville, Md., cat. nos. CRL 1934 and CRL 1821) and RW4 (Genome Systems, Inc., St. Louis, Mo., catalog no. ESVJ-1182). Transfection of the KO construct can be accomplished by, e.g., electroporation, microinjection, or calcium phosphate treatment.

Offspring that are positive for the IL-19KO or IL-24KO construct will typically be heterozygous, although some homozygous knockouts may exist. If homozygous knockout mammals are desired, they can be prepared by crossing those heterozygous offspring believed to carry the knockout construct in their germ line to each other.

Methods of molecular biology for producing cytokine KO constructs are described, see, e.g., Power (2003) J. Immunol. Methods 273:73-82; Sauer (1998) Methods 14:381-392; Copeland, et al. (2001) Nature Revs. 2:769-779; Voorhoeve and Agami (2003) Trends Biotechnol. 21:2-4; Walke, et al. (2001) Curr. Opinion Biotechnol. 12:626-631; Galli-Taliadoros, et al. (1995) J. Immunol. Methods 181:1-15; Lovik (1997) Toxicology 119:65-76; Charreau, et al. (1996) Transgenic Res. 5:223-234; te Riele, et al. (2001) Methods Mol. Biol. 158:251-262; Osada and Maeda (1998) Methods Mol. Biol. 110:79-92; Ravirajan and Isenberg (2002) Lupus 11:843-849; van der Weyden, et al. (2002) Physiol. Genomics 11:133-164.

Methods for using stem cells to produce cytokine KO mice are described, see, e.g., U.S. Pat. No. 6,087,555 issued to Dunstan, et al.; Potten (ed.) (1997) Stem Cells, Academic Press, San Diego, Calif.; Sell (ed.) (2003) Stem Cells Handbook; Human Press, Totowa, N.J.; Marshak, et al. (eds.) (2002) Stem Cell Biology, Cold Spring Harbor Laboratory Press, Cold Spring Harbor, N.Y.; Turksen (ed.) (2001) Embryonic Stem Cells; Human Press, Totowa, N.J.; Durum, et al. (eds.) (1998) Cytokine Knockouts, Humana Press, Totowa, N.J.; Jacob (ed.) (1994) Overexpression and Knockout of Cytokines in Transgenic Mice, Academic Press, San Diego, Calif.; Tymms and Kola (eds.) (2001) Gene Knockout Protocols, Humana Press, Totowa, N.J.

Expression of IL-19 by the IL-19KO mouse and expression of IL-24 by the IL-24KO mouse are typically less than 60%, more typically less than 30%, generally less than 15%, preferably under 5%, more preferably under 2%, and most preferably under 1%, that from a suitable wild type mouse.

In comparing a KO mouse with a suitable wild type mouse, it is preferable that the KO mouse and wild type mouse be as closely related as possible, e.g., the wild type mouse should be of the same strain as the mouse used as the source of stem cells in preparing the KO mouse.

The invention contemplates methods of using IL-19KO mice or IL-24KO mice for the screening and testing of diagnostic, pharmaceutical, and therapeutic agents. For screening purposes, the IL-19KO or IL-24KO animals can be treated with various analogues of IL-19 or IL-24, in order to determine which analogue functions most like, or superior to, administered IL-19 or IL-24. An endpoint in the screening assay can be a change in macrophage activity or concentration to a predetermined level, a change in dendritic cell activity or concentration to a predetermined level, an increase in airway hyperreactivity to a predetermined level, e.g., the level found in a wild type mouse or suitable control mouse.

### IV. Agonists, Antagonists, and Binding Compositions.

Antibodies and binding compositions derived from an antigen-binding site of an antibody are provided. These include humanized antibodies, human antibodies, monoclonal or polyclonal antibodies, binding fragments, e.g., Fab, F(ab)₂, and Fv fragments, and engineered versions thereof. “Derived” includes derived by chemical modification of an antibody, by recombinant modification of an antibody, as well as derived by computer modeling of the binding composition after the structural features of the antibody. The antibody or binding composition can be agonistic or antagonistic. Antibodies that simultaneously bind to a ligand and receptor, to both subunits of a dimeric ligand, or to both subunits of a dimeric receptor, are contemplated. Also encompassed are small molecule peptide mimetics of IL-19, IL-24, anti-IL-19 antibody, and anti-IL-24 antibody.

Antibodies will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al (2001) Crit. Rev. Oncol. Hematol. 38:17-23; Carnahan, et al. (2003) Clin. Cancer Res. (Suppl.) 9:3982s-3990s).

The invention provides agonists and antagonists of IL-19, e.g., muteins and naturally occurring variants of IL-19, binding compositions, such as anti-IL-19 antibodies, soluble versions of IL-19 receptor polypeptides, and binding compositions and antibodies to IL-19 receptor polypeptides. Also provided are agonists and antagonists of IL-24, e.g., muteins and naturally occurring variants of IL-24, binding compositions, such as anti-IL-24 antibodies, soluble versions of IL-24 receptor polypeptides, and binding compositions and antibodies to IL-24 receptor polypeptides.

Antibodies to human IL-19 (SEQ ID NO:2) can be prepared. Regions of increased antigenicity in human IL-19 include KRAIQAKD (amino acids 45-52 of SEQ ID NO:2); TKNLLA (78-83 of SEQ ID NO:2); KDHQ (91-94 of SEQ ID NO:2); and KTLR (116-119 of SEQ ID NO:2). Also contemplated are antibodies to variants of hIL-19, see, e.g., Genbank Accession Nos. NP_{—}758846; NP_{—}443104; and AAH09681.

Antibodies to human IL-24 (SEQ ID NO:4) can be prepared. Regions of increased antigenicity in human IL-24 include AVKD (amino acids 48-51 of SEQ ID NO:4); SARLLQ (61-66 of SEQ ID NO:4); LVHTLL (81-86 of SEQ ID NO:4); LKTVFKNYHN (90-99 of SEQ ID NO:4); DSAHRR (137-142 of SEQ ID NO:4); and RRAFKQLDVEAALTKAL (147-163 of SEQ ID NO:4). Also contemplated are antibodies to variants of hIL-24, see, e.g., Genbank Accession Nos. NP_{—}715639 and NP_{—}037503.

Antibodies to IL-19 receptor polypeptides and IL-24 receptor polypeptides, i.e., IL-20R1 (a.k.a. crf2-8; zcytor7); IL-20R2 (a.k.a. crf2-11; DIRS1); and IL-22R, can be prepared. Anti-IL-22R antibodies are available (Dumoutier, et al. (2001) J. Immunol. 167:3545-3549). Regions of increased antigenicity of human IL-20RA include amino acids 79-85; 103-114; 155-163; 200-205; 234-243; and 278-287 of GenBank Accession No. NM_{—}014432. Regions of increased antigenicity of human IL-20RB include amino acids 49-51; 77-81; 111-116; 127-130; 147-152; and 233-237, of GenBank Accession No. AAQ47003. Antigenicity was determined by the Welling plot of Vector NTI® Suite (Informax, Inc, Bethesda, Md.).

Monoclonal, polyclonal, and humanized antibodies can be prepared, see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205-6213; He, et al. (1998) J. Immunol. 160:1029-1035; Tang, et al. (1999) J. Biol. Chem. 274:27371-27378; Li, et al. (2002) Immunol. Revs. 190:53-68; Sato, et al. (1994) Mol. Immunol. 31:371-381; Morea, et al. (2000) Methods 20:267-279.

A humanized antibody contains the amino acid sequences from six complementarity determining regions (CDRs) of the parent mouse antibody, which are grafted on a human antibody framework. Alternatives to humanization include use of fully human antibodies, as well as human antibody libraries displayed on phage or human antibody libraries contained in transgenic mice, see, e.g., Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nat. Biotechnol. 17:397-399.

Humanized antibodies, chimeric antibodies, single chain antibodies, single domain antibodies, bispecific antibodies, and peptide mimetics of antibodies are described (see, e.g., Maynard and Georgiou (2000) Annu. Rev. Biomed. Eng. 2:339-376; Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) New Engl. J. Med. 148:1547-1553; Casset, et al. (2002) Biochem. Biophys. Res. Commun. 307:198-205; U.S. Pat. Nos. 5,932,448; 5,532,210; 6,129,914; 6,133,426; 4,946,778).

Purification of antigen is not necessary for the generation of antibodies. Immunization can be performed by DNA vector immunization, see, e.g., Wang, et al. (1997) Virology 228: 278-284. Alternatively, animals can be immunized with cells bearing the antigen of interest followed by hybridoma production, see, e.g., Meyaard, et al. (1997) Immunity 7:283-290; Wright, et al. (2000) Immunity 13:233-242; Preston, et al. (1997) Eur. J. Immunol. 27:1911-1918; Kaithamana, et al. (1999) New Engl. J. Med. 163:5157-5164.

Antibody/antigen binding properties can be measured, e.g., by surface plasmon resonance or enzyme linked immunosorbent assay (ELISA) (Neri, et al. (1997) Nat. Biotechnol. 15:1271-1275; Jonsson, et al. (1991) Biotechniques 11:620-627; Hubble (1997) Immunol. Today 18:305-306). The antibodies of this invention can be used for affinity chromatography in isolating the antibody's target antigen and associated bound proteins (Wilchek, et al. (1984) Meth. Enzymol. 104:3-55).

Soluble receptors comprising the extracellular domains of IL-19 or IL-24 receptor polypeptides are provided. The extracellular domains of IL-20R1 (a.k.a. crf2-8), IL-20R2 (a.k.a. crf2-11), and IL-22R (a.k.a. crf2-9; zcytor 11) are disclosed, see, e.g., Kotenko (2002) Cytokine Growth Factor Revs. 13:223-240; Kotenko, et al. (2001) J. Immunol. 166:7096-7103; Dumoutier, et al. (2001) J. Immunol. 166:7090-7095. Soluble receptors can be prepared and used according to standard methods, see, e.g., Jones, et al. (2002) Biochim. Biophys. Acta 1592:251-263; Prudhomme, et al. (2001) Expert Opinion Biol. Ther. 1:359-373; Fernandez-Botran (1999) Crit. Rev. Clin. Lab Sci. 36:165-224.

Nucleic acid binding compounds are provided for diagnostic or kit uses, e.g., a nucleic acid encoding SEQ ID NOs:2 or 4, or an antigenic fragment, thereof, for use, e.g., as PCR primers, hybridization primers, and molecular beacons.

### IV. Therapeutic Compositions, Methods.

The invention provides IL-19, anti-IL-19 antibodies, IL-24, and anti-IL-24 antibodies for use, e.g., in the treatment of inflammatory and autoimmune disorders. Nucleic acids are also provided for these therapeutic uses, e.g., nucleic acids encoding SEQ ID NOs:2 or 4, an antigenic fragment thereof, the corresponding anti-sense nucleic acids, and hybridization products thereof. The invention also provides compositions for RNA interference, see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189.

To prepare pharmaceutical or sterile compositions including an agonist or antagonist of IL-19, an agonist or antagonist of IL-24, e.g., the cytokine, cytokine analogue or mutein, or antibody thereto, is admixed with a pharmaceutically acceptable carrier or excipient which is preferably inert, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of, e.g., lyophilized powders, slurries, aqueous solutions or suspensions, see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available, see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602.

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg, see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

An effective amount for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK.

Typical veterinary, experimental, or research subjects include monkeys, dogs, cats, rats, mice, rabbits, guinea pigs, horses, and humans.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

Methods for co-administration or treatment with a second therapeutic agent, e.g., a cytokine, steroid, chemotherapeutic agent, antibiotic, or radiation, are well known in the art, see, e.g., Hardman, et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa. An effective amount of therapeutic will decrease the symptoms typically by at least 10%; usually by at least 20%; preferably at least about 30%; more preferably at least 40%, and most preferably by at least 50%.

### X. Kits and Diagnostic Reagents.

This invention provides polypeptides of IL-19 and IL-24, fragments thereof, nucleic acids of IL-19 and IL-24, and fragments thereof, in a diagnostic kit, e.g., for the diagnosis of asthma, airway hyperreactivity, dendritic cell-mediated disorders, and macrophage-mediated disorders. Also provided are binding compositions, including antibodies or antibody fragments, for the detection of IL-19, IL-24, and metabolites and breakdown products thereof. Typically, the kit will have a compartment containing either a IL-19 or IL-24 polypeptide, or an antigenic fragment thereof, a binding composition thereto, or a nucleic acid, such as a nucleic acid probe, primer, or molecular beacon, see, e.g., Rajendran, et al. (2003) Nucleic Acids Res. 31:5700-5713; Cockerill (2003) Arch. Pathol. Lab. Med. 127:1112-1120; Zammatteo, et al. (2002) Biotech. Annu. Rev. 8:85-101; Klein (2002) Trends Mol. Med. 8:257-260.

A method of diagnosis can comprise contacting a sample from a subject, e.g., a test subject, with a binding composition that specifically binds to a polypeptide or nucleic acid of IL-19 (SEQ ID NOs:1 or 2); IL-24 (SEQ ID NOs:3 or 4); IL-20R1; IL-20R2; or IL-22R. The method can further comprise contacting a sample from a control subject, normal subject, or normal tissue or fluid from the test subject, with the binding composition. Moreover, the method can additionally comprise comparing the specific binding of the composition to the test subject with the specific binding of the composition to the normal subject, control subject, or normal tissue or fluid from the test subject. Expression or activity of a test sample or test subject can be compared with that from a control sample or control subject. A control sample can comprise, e.g., a sample of non-affected or non-inflamed tissue in a patient suffering from an immune disorder. Expression or activity from a control subject or control sample can be provided as a predetermined value, e.g., acquired from a statistically appropriate group of control subjects.

The kit may comprise, e.g., a reagent and a compartment, a reagent and instructions for use, or a reagent with a compartment and instructions for use. The reagent may comprise an agonist or antagonist of IL-19 or IL-24, or an antigenic fragment thereof, a binding composition, or a nucleic acid in a sense and/or anti-sense orientation. A kit for determining the binding of a test compound, e.g., acquired from a biological sample or from a chemical library, can comprise a control compound, a labeled compound, and a method for separating free labeled compound from bound labeled compound. The control compound can comprise a segment of SEQ ID NOs:2 or 4, or a nucleic acid of SEQ ID NOs:1 or 3 that encodes a segment of SEQ ID NOs:2 or 4. The segment can comprise zero, one, two, or more antigenic fragments.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, fluorescent dyes, electron-dense reagents, substrates, epitope tags, or enzymes, e.g., as used in enzyme-linked immunoassays, or fluorettes (Rozinov and Nolan (1998) Chem. Biol. 5:713-728).

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals, see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J. Med. 168:883-889. Various assay formats exist, such as radioimmunoassays (RIA), ELISA, and lab on a chip (U.S. Pat. Nos. 6,176,962 and 6,517,234).

### XI. Uses.

The invention provides methods for the treatment and diagnosis of a number of disorders, including inflammatory disorders of the airways, airway hyperreactivity, and pulmonary fibrosis. Provided are methods for the treatment and diagnosis of pulmonary conditions involving the lung parenchyma, cuboidal epithelium, type I alveolar epithelium, type II alveolar epithelium, alveolar spaces, or interstitium. Also provided are methods of treatment and diagnosis involving samples of the BAL, sputum, and biopsies (see, e.g., Doerschuk (2000) Respir. Res. 1:136-140; Cosio, et al. (1999) Am. J. Respir. Crit. Care Med. 160:S21-S25; Cosio, et al. (2002) Chest 121:160S-165S).

The invention provides methods for treating and diagnosing disorders that are modulated by monocytes or macrophages, dendritic cells, neutrophils, eosinophils, mast cells, T cells, including TH1-type cells and TH2-type cells, B cells, epithelial cells; or endothelial cells. These disorders include, e.g., disorders of the airways, parenchyma, or alveoli, including asthma, bronchitis, bronchiolitis, status asthmaticus, and COPD. Provided are methods to modulate cells that mediate airway remodeling, e.g., epithelial cells, smooth muscle cells, and other stromal cells (Kumar (2001) Pharmacol. Therapeutics 91:93-104).

Provided are methods of treatment and/or diagnosis of fibrosis and granulomas. Fibrosis and granulomas are features of interstitial lung disorders, e.g., idiopathic pulmonary fibrosis, eosinophilic granuloma, and hypersensitivity pneumonitis. These disorders involve activated alveolar epithelial cells, activated macrophages, and/or lymphocytes (Kamp (2003) Chest 124:1187-1189; Patel, et al. (2001) J. Allergy Clin. Immunol. 108:661-670).

Disorders treated and/or diagnosed by the present invention are not limited to disorders of the airways, but also include, e.g., inflammatory bowel disorders (IBD), rheumatoid arthritis or synovitis, psoriasis, graft and transplant rejection, systemic lupus erythematosus (SLE), inflammation of the central nervous system, e.g., multiple sclerosis, sepsis, atherosclerosis, diabetes mellitus, as well as innate immunity and acquired immunity to pathogens, such as bacteria, parasites, and viruses.

Provided are agonists or antagonists of IL-19 or IL-24 to modulate disorders and conditions dependent on monocytes or macrophages, see, e.g., Chedevergne, et al. (2000) Arch. Dis. Child. 82 (suppl.2) II6-9; Jeffery (1999) Clin. Exp. Allergy 29 (suppl. 2):14-26; Essadki, et al. (1998) Eur. Radiol. 8:1674-1676; Jeffery (2000) Chest 117 (suppl. 1):251S-260S; Berrebi, et al. (2003) Gut 52:840-846; Hibi, et al. (2003) J. Gastroenterol. 38 (suppl.15):36-42; Watanabe, et al. (2003) Dig. Dis. 48:408-414; Shimizu, et al. (2002) Histochem. Cell Biol. 118:251-257; Zander, et al. (1999) J. Heart Lung Transplant. 18:646-653; Slegers, et al. (2003) Curr. Eye Res. 26:73-79; Milne, et al. (1998) Transplantation 15:671-673; Steinbach, et al. (2000) Ann. Rheum. Dis. 59:283-288; Schwartz (2003) J. Cereb. Blood Flow Metab. 23:385-394; Underhill (2003) Eur. J. Immunol. 33:1767-1775; Openshaw (2002) Respir. Res. 3 (suppl. 1):S15-S20; Qian, et al. (1999) Am. J. Pathol. 155:1293-1302; Brettschneider, et al. (2002) J. Neuroimmunol. 133:193-197; Klebl, et al. (2001) J. Pathol. 195:609-619; Szekanecz, et al. (2001) Curr. Rheumatol. Rep. 3:53-63; Boehncke, et al. (1995) Am. J. Dermatopathol. 17:139-144; Sica, et al. (2002) Int. Immunopharmacol. 2:1045-1054;

The invention provides an agonist or antagonist comprising a binding composition derived from the antigen binding site of an antibody, where the agonist or antagonist specifically binds to a polypeptide of IL-19 (SEQ ID NO:2); IL-24 (SEQ ID NO:4); IL-20R1; IL-20R2; or IL-22R. Also provided is an agonist or antagonist comprising a nucleic acid that specifically binds to a nucleic acid of IL-19 (SEQ ID NO:1); IL-24 (SEQ ID NO:3); IL-20R1; IL-20R2; or IL-22R.

IL-19 was found to modulate adipose tissue thickness, that is, IL-19KO mice showed an increase in thickness of adipose tissue, as determined by histological measurements. Cytokines and cytokine-like compounds, e.g., leptin and leptin receptor, are established regulators of adipose tissue metabolism, obesity, diabetes, and perhaps wound healing or skin repair. Adipose tissue thickness and obesity can be assessed by methods of histology and anthropometry (see, e.g., Veniant and LeBel (2003) Curr. Pharm. Des. 9:811-818; Sandoval and Davis (2003) J. Diabetes Complic. 17:108-113; Coppack (2001) Proc. Nutr. Soc. 60:349-356; Fantuzzi and Faggioni (2000) J. Leukocyte Biol. 68:437-446; Goren, et al. (2003) Diabetes 52:2821-2832; Piemonti, et al. (2003) Diabetes Care 26:2883-2999; Taggart, et al. (1967) Brit. J. Nutr. 21:439-451; Lee and Nieman (1996) Nutritional Assessment, 2^{nd} ed., Mosby Year Book, St. Louis, Mo.; Rolland-Cachera, et al. (1997) Am. J. Clin. Nutr. 65:1709-1713; Jen, et al. (1985) Int. J. Obes. 9:213-224).

The broad scope of this invention is best understood with reference to the following examples, which are not intended to limit the inventions to the specific embodiments.

### EXAMPLES

### I. General Methods.

Some of the standard methods are described or referenced, e.g., in Maniatis, et al. (1982) Molecular Cloning, A Laboratory Manual, Cold Spring Harbor Press, Cold Spring Harbor, N.Y.; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987) Current Protocols in Molecular Biology and supplements, Greene/Wiley, New York. Methods for protein purification include, e.g., column chromatography, electrophoresis, centrifugation, immunoprecipitation, and cloning and expression by vectors and cells, see, e.g., Amersham Pharmacia Biotech (2003) Catalogue, Piscataway, N.J.; Invitrogen (2003) Catalogue, Carlsbad, Calif.; Sigma-Aldrich (2003) Catalogue, St. Louis, Mo.

Methods for flow cytometry, including fluorescence activated cell sorting (FACS), are available, see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Cell counting can be accomplished with the aid of beads or microspheres, e.g., Caltag® counting beads (Caltag Labs, Burlingame, Calif.) and Perfectcount® (Exalpha Biologicals, Watertown, Mass.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described, see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y. Methods relating to bronchoalveolar lavage and lung challenge tests, e.g., involving Aspergillus, histamine, and methacholine, are available. Macrophages, neutrophils, and eosinophils participate in host defense against Aspergillus, see, e.g., Kurup and Grunig (2002) Mycopathologia 153:165-177; Schuh, et al. (2002) EMBO J. 16:1313-1315; Wark, et al. (2000) Eur. Respir. J. 16:1095-1101; Philippe, et al. (2003) Infect Immun. 71:3034-3042, Feller-Kopman and Ernst (2003) Semin. Respir. Infect. 18:87-94; Marr, et al. (2002) Infect. Dis. Clin. North Am. 16:875-894; Kurup and Grunig (2002) Mycopathologia 153:165-177; Joos (2003) Curr. Opin. Pharmacol. 3:233-238; Cockcroft, et al. (2001) Chest 120:1857-1860; Brusasco and Crimi (2001) Allergy 56:1114-1120; O'Byrne and Inman (2000) J. Asthma 37:293-302; Buckingham and Hansell (2003) Eur. Radiol. 13:1786-17800; Kurup, et al. (2002) Int. Arch. Allergy Immunol. 129:181-188; Wheat, et al. (2002) Semin. Respir. Infect. 17:158-181.

Methods for using animal models, e.g., knockout mice, and cell-based assays for the testing, evaluation, and screening of diagnostic, therapeutic, and pharmaceutical agents are available, see, e.g., Car and Eng (2001) Vet. Pathol. 38:20-30; Kenyon, et al. (2003) Toxicol. Appl. Pharmacol. 186:90-100; Deurloo, et al. (2001) Am. J. Respir. Cell Mol. Biol. 25:751-760; Zuberi, et al. (2000) J. Immunol. 164:2667-2673; Temelkovski, et al. (1998) Thorax 53:849-856; Horrocks, et al. (2003) Curr. Opin. Drug Discov. Devel. 6:570-575; Johnston, et al. (2002) Drug Discov. Today 7:353-363.

Software packages for determining, e.g., antigenic fragments, signal and leader sequences, protein folding, and functional domains, are available, see, e.g., Vector NTI® Suite (Informax, Inc., Bethesda, Md.); GCG Wisconsin Package (Accelrys, Inc., San Diego, Calif.), and DeCypher® (TimeLogic Corp., Crystal Bay, Nev.); Menne, et al. (2000) Bioinformatics 16:741-742. Public sequence databases were also used, e.g., from GenBank and others.

### II. Knockout Mice.

IL-19 knockout (IL-19KO) mice were made using the general methods described by Chensue, et al. (2001) J. Exp. Med. 193:573-584. The parent mouse strain was 129 SvEv. In brief, a neomycin cassette was inserted into an exon of the IL-19 gene, resulting in a gene product harboring the neomycin gene. Utilizing a cDNA probe for mouse IL-19, a BAC clone containing the entire IL-19 locus was identified. A 12 kilobase (kb) EcoRI fragment containing exons 1-5 was subcloned. Utilizing this plasmid a targeting vector with a 1,752 base pairs (bp) BamHI fragment (5′ region of homology) and a of 4,044 bp XmnI fragment (3′ region of homology) was constructed. The targeted locus had exons 3 and 4 replaced with a floxed Neo cassette driven by a HSV TK promoter in the opposite direction of IL-19. The properly targeted locus was identified by digesting genomic DNA from Neo resistant ES cell clones with NcoI and hybridizing with a 372 bp probe generated using PCR on the 12 kb EcoRI fragment with primers 1: AAGTGATTTCGTTTGGCTG (SEQ ID NO:5) and 2: TGTCTGGTAAGATCCTATC (SEQ ID NO:6). Verification of the 3′ arm integration was done by digesting genomic DNA from Neo resistant Embryonic Stem (ES) cell clones with EcoRI and hybridizing with a 322 bp probe generated using PCR on the 12 kb. EcoRI fragment with primer no.1: CACCATCAGCAGCTTGGTC (SEQ ID NO:7), and primer no.2: TAATCCTCATGCAGCTCTG (SEQ ID NO:8).

IL-19 targeted ES cell clones were generated. ES cells were electroporated with the linearized targeting vector as described (Joyner (1993) Gene Targeting: A Practical Approach, IRL Press, Oxford, UK). Six out of 1367 G418-resistant clones were detected as homologous recombinants by Southern analysis with a 5′ and a 3′ external probe. The single-copy replacement of the targeting construct was confirmed by Southern analysis using a neo probe.

Chimeric, heterozygous and homozygous mice were generated. Two targeted ES clone were injected into 3.5 dpc C57BL/6 (CRL) blastocysts, which were then transferred into CD1 pseudo pregnant female mice at day 2.5 dpc for gestation. Chimeric male progeny were bred with C57BL/6 (CRL) females and germline transmission was recognized by the agouti coat color in the offspring. Heterozygous mice inheriting the mutated allele from the father were detected by PCR or Southern analysis of DNA. Progeny from intercrosses of heterozygotes were genotyped by PCR or Southern analysis of DNA.

IL-24KO mice were prepared using the general methods described by Lemckert, et al. (1997) Nucleic Acids Res. 25:917-918. The parent mouse strain was C57BL/6. In brief, a neomycin cassette was inserted into an exon of IL-24 and then flipped out, resulting in deletion of the exon and the neomycin gene. Further details on the Cre-Lox method used in preparing the IL-24KO are described (Ruuls, et al. (2001) Immunity 15:533-543). Utilizing a cDNA probe for mouse IL-24, a BAC clone containing the entire IL-24 locus was identified. An IL-24 locus subclone of 12,763 bp was sequenced and found to contain five IL-24 exons. A targeting vector containing a 1.6 kb StuI and HpaI fragment (5′ region of homology) and a 5.34 kb XbaI and PacI fragment (3′ region of homology) was constructed. The targeted locus has exons 1 and 2 replaced with a floxed Neo cassette being driven by a HSV TK promoter in the opposite direction of IL-24. The properly targeted locus was identified by digesting genomic DNA from Neo resistant ES cell clones with HindIII and hybridizing with a probe from region 1220-1600 bp from the IL-24 locus subclone. Verification of the 3′ arm integration was done by digesting genomic DNA from Neo resistant ES cell clones with HpaI and hybridizing with a probe from region 11050-11391 bp from the IL-24 locus subclone.

### III. Cellular Analysis of Bronchoalveolar Lavage Fluid (BAL).

Aspergillus fumigatus (Asp; Asp-treatment; Asp-challenge) induces a number of the signs of asthma and of other immune disorders of the lungs, e.g., airway hyperreactivity, goblet cell hyperplasia, mucus overproduction, and eosinophilic airway inflammation. Mice were treated intranasally with Aspergillus antigen or with phosphate buffered saline (PBS) at intervals of four days, at t=1, 5, 9, 12, and 16 days (Table 1). Cells were identified in the BAL from wild type mice, IL-19KO mice, and IL-24KO mice. All of the cell counts represent numbers of cells per bead. BAL samples were provided with 32,000 fluorescent beads to facilitate counting (Table 1).

Asp-treatment modulated the total number of BAL cells, where treatment increased the BAL cells in all three genotypes of mice: the wild type, IL-19KO, and IL-24KO mice. As shown in Table 1, with Asp-treatment, the number of cells in the IL-19KO BAL and IL-24KO BAL was somewhat lower than in the wild type BAL (Table 1). Asp-treatment also increased the CD11c⁺ MHC low cells in all three genotypes of mice, where the number of CD11c⁺ MHC low cells in the Asp-treated IL-19KO mouse BAL was lower than in the Asp-treated wild type BAL (Table 1). The CD11c⁺ MHC low cells were mainly DCs, monocytes, and macrophages.

Responses in B cell number and B cell expression of MHC Class II differed in the IL-19KO and the IL-24KO, relative to the wild type mouse. With Asp-treatment, B cell number was low in the IL-19KO mice and high in the IL-24KO mice, relative to the B cell count in the wild type mouse (Table 1). With Asp-treatment, MCH Class II expression increased on B cells from the IL-19KO, and decreased on B cells from the IL-24KO (Table 1).

Macrophage and DC counts were reduced in the IL-19KO mouse, relative to counts in the wild type mouse (Table 1).

Regarding the wild type mice, two types of wild type mice were tested for percentage of cells expressing CD11c and MHC Class II. These two types were female 129 SvEv (12 weeks old) and female C57 BL/6 (7 weeks old) mice. Expression of CD11c in IL-19KO mice was reduced, when compared to either of the above two types of wild type mice.

TABLE 1
Cells identified in BAL.

| | | | | | |
|---|---|---|---|---|---|
| Wild type | IL-19KO | IL-24KO | Wild type | IL-19KO | IL-24KO |
| (PBS) | (PBS) | (PBS) | (Asp) | (Asp) | (Asp) |

Total cells in gate (cells/bead)

| | | | | | |
|---|---|---|---|---|---|
| 3.8 | 12.93 | 0.63 | 57.0 | 47.7 | 35.9 |

CD11c⁺ MHC II low cells (cells/bead)

| | | | | | |
|---|---|---|---|---|---|
| 0.251 | 0.046 | 0.208 | 0.795 | 0.499 | 0.783 |

B cells (cells/bead)

| | | | | | |
|---|---|---|---|---|---|
| Low | Low | Low | 0.42 | 0.169 | 1.33 |

MHC class II expression on B cells
(fluorescence; relative to wild type)

| ND | ND | ND | 161.9 | 204 | 120.4 |
|---|---|---|---|---|---|
| Macrophages (relative to 100%) | | | Macrophages (relative to control) | | |
| 100% | 33% | ND | Control | reduced | ND |
| Dendritic cells (relative to 100%) | | | Dendritic cells (relative to control) | | |
| 100% | 50% | ND | Control | reduced | ND |

Proportion of dendritic cells expressing B220

| | | | | | |
|---|---|---|---|---|---|
| Control | increased | ND | ND | ND | ND |

ND means not determined.

Histological analysis demonstrated that Asp-treated IL-19KO mouse lungs were less inflamed in comparison to the two Asp-treated wild types, in particular parenchymal inflammation. Both Asp-treated wild type and Asp-treated IL-19KO mice showed goblet cell hyperplasia. IL-24KO mice had a more structured inflammation as compared to the wild type, i.e., they had lymph follicle type structures in the lungs, a finding consistent with FACS data showing increased B cells in Asp-treated IL-24KO mouse BAL. Asp-treated wild type and Asp-treated IL-24KO lungs showed similar inflammation and goblet cell hyperplasia, according to histological analysis. The histology also showed that C57 BL/6 wild type mice were somewhat less sensitive to Aspergillus than the 129 SvEv wild type mice, i.e., the 129 SvEv wild type mice showed more Asp-induced inflammation.

The IL-19KO mice were also found to have increased adipose tissue deposits, as determined by assays of adipose tissue in the skin.

### IV. Airway Hyperreactivity After Challenge.

Airway hyperreactivity was measured after: control treatment with PBS; challenge with Aspergillus only; treatment with methacholine only; or treatment with both methacholine and Aspergillus (Table 2). Methacholine is used for assessing airway hyperreactivity in human patients as well as in experimental animals (Gronke, et al. (2002) Clin. Exp. All. 32:57-63; Obase, et al. (2003) Allergy 58:213-220). Airway hyperreactivity was tested by the Penh method (see, e.g., Kenyon, et al. (2003) Toxicol. Applied Pharmacol. 191:2-11; Hantos, et al. (2002) J. Appl. Physiol. 93:1196-1197; Hamelmann, et al. (1997) Am. J. Respir. Critical Care Med. 156:766-775).

The challenge protocol involved methacholine doses of 4.17 or 10.0 mg/ml. The dosing protocol consisted of three cycles, each cycle comprising three steps, i.e., an aerosol phase (3.0 min), drying phase (0.5 min), and recording phase (5 min). Each cycle of three steps lasted 8.5 min. Three separate doses of an identical amount of methacholine, in the three successive cycles (MCh 1; MCh 2; and MCh 3), were administered to each mouse. The test was conducted with 0, 4.17, or 10 mg/ml of methacholine, followed by assessments of airway hyperreactivity. The reactivity with the PBS was set to 100% (Table 2). The Aspergillus treatment protocol involved intranasal Aspergillus fumigatus antigen at intervals of four days, i.e., at t=1, 5, 9, 12, and 16 days.

Asp-treated IL-19KO mice showed less hyperreactivity than Asp-treated wild type mice. Similarly, Asp-treated KO mice showed less hyperreactivity than Asp-treated wild type mice.

The IL-19KO exhibited reduced hyperreactivity in response to methacholine alone and to methacholine plus Aspergillus (Table 2). Similarly, the IL-24KO also had reduced hyperreactivity in response to methacholine alone and to methacholine plus Aspergillus (Table 2). Mice with a hyperreactivity value of 300% or greater, after methacholine treatment exhibited dyspnea.

Without Aspergillus challenge, airway hyperreactivity appeared the same in wild type and IL-24KO mice. However, with the challenge, hyperreactivity was lower in the IL-24KO mice than in the wild type mice. Because of the established role of B cell to T cell interactions in airway hyperreactivity, the reduced expression of MHC class II on B cells in the IL-24KO mice can account for the reduced hyperreactivity in these animals (Waldburger, et al. (2001) J. Exp. Med. 194:393-406; Pai, et al. (2002) J. Immunol. 169:1326-1333).

TABLE 2
Airway hyperreactivity after methacholine treatment.

| | | | | | |
|---|---|---|---|---|---|
| Wild type | IL-19KO | IL-24KO | Wild type | IL-19KO | IL-24KO |
| (PBS) | (PBS) | (PBS) | (Asp) | (Asp) | (Asp) |

PBS (hyperreactivity set at 100%)

| | | | | | |
|---|---|---|---|---|---|
| 100% | 100% | 100% | 100% | 100% | 100% |

Methacholine dose (4.17 mg/ml)

| | | | | | |
|---|---|---|---|---|---|
| 186.8% | 146.5% | 150.5% | 450.6% | 247.5% | 205.0% |

Methacholine dose (10.0 mg/ml)

| | | | | | |
|---|---|---|---|---|---|
| 309.0% | 239.4% | 158.4% | ND | 312.2% | 343.4% |

### V. Analysis of Cells in Lymph Nodes.

Mice were treated intranasally with Aspergillus fumigatus antigen at intervals of four days, i.e., at t=1, 5, 9, 12, and 16 days. Cells from lymph nodes from wild type mice, IL-19KO mice, IL-24KO mice were identified. The mice were treated with Aspergillus (Asp), as indicated (Table 3). Lymph node samples were provided with 64,000 beads to facilitate cell counting. Lymph node B cell number increased in the IL-24KO mouse, relative to the wild type (data not shown), and in the Asp-treated IL-24KO mouse relative to the Asp-treated wild type (Table 3). MHC Class II expression was measured by fluorescence, and is disclosed in arbitrary units of fluorescence. The fluorescence intensity is relative to the number of molecules expressed by the cell.

TABLE 3
Cells identified in mouse lymph nodes.

| Wild type | IL-19KO | IL-24KO | Wild type | IL-19KO | IL-24KO |
|---|---|---|---|---|---|
| (PBS) | (PBS) | (PBS) | (Asp) | (Asp) | (Asp) |
| B cells (relative to 100%) | | | B cells (cells/bead) | | |
| 100% | ND | increased | 8.75 | 6.24 | 32.99 |

B cell expression of MHC class II (fluorescence)

| | | | | | |
|---|---|---|---|---|---|
| ND | ND | ND | 161.93 | 204 | 120.48 |

ND means not determined.

In addition to the changes disclosed in Table 3, there was a trend towards reduced numbers of DCs in lymph nodes of IL-19KO mice, as compared to wild type mice. There were also reduced numbers of DCs in lymph nodes of Asp-treated IL-19KO mice, as compared to Asp-treated wild type mice. Lymph node macrophages were also reduced in Asp-treated IL-19KO mice, as compared with Asp-treated wild type mice.

### VI. PCR Analysis of IL-19, IL-24 Distribution.

Expression and distribution of IL-19 and IL-24 in human and mouse cells and tissues were determined by Taqman® real time PCR (PE Applied Biosystems, Foster City, Calif.), where the results are relative to ubiquitin expression (Tables 4 and 5). Ubiquitin expression is set to one (1.0). In particular, IL-19 and IL-24 expression increased in response to monocyte/macrophage or dendritic cell activation or to antigen challenge to the lung (Tables 4 and 5).

TABLE 4
Expression of IL-19 by Taqman ® analysis, relative to ubiquitin (1.0).
Human cells

| | |
|---|---|
| Monocyte, resting | 0.0 |
| Monocyte, LPS activated | 121 |
| Monocyte, anti-CD3, anti-CD28 activated | 292 |
| Dendritic cell, resting | 0.0 |
| Dendritic cell, CD40 ligand activated | 24 |

Mouse cells

| | |
|---|---|
| TH2 cell fresh | 0.0 |
| TH2 cell activated, phorbol ester, ionomyin | 52 |
| Macrophage, resting | 2 |
| Macrophage, activated LPS | 323 |

TABLE 5
Expression of IL-24 by Taqman ® analysis, relative to ubiquitin (1.0).
Mouse cells and tissues

| | |
|---|---|
| BALB/c T cell resting | 0.0 |
| BALB/c T cell TH1 fresh | 54 |
| BALB/c T cell TH1 activated, phorbol ester, ionomycin | 574 |
| BALB/c T cell TH2 fresh | 5754 |
| BALB/c T cell TH2 activated, phorbol ester, ionomycin | 13655 |
| Endothelial cell, resting | 0.0 |
| Endothelial cell, activated TNFalpha | 100 |
| Lung untreated | 3 |
| Lung Aspergillus challenged, intranasal | 33 |
| Lung Nippostrongulus infected | 10 |

### VII. IL-19 Modulates Adipose Tissue Thickness.

IL-19 knockout provokes an increase in adipose tissue thickness in the L-19KO mouse. The thickness of the layer of adipose tissue between the dermis and panniculus carnosus increased 2-3 fold, as determined by histological methods. The invention provides methods of modulating adipose tissue thickness, adiposity, obesity, and diabetes, by an agonist or antagonist of IL-19 or IL-24. The invention provides an IL-19 or IL-24 agonist, e.g., IL-19 polypeptide or a nucleic acid encoding IL-19, for the treatment of obesity, adiposity, or diabetes. Also provided are methods for the diagnosis of obesity, adiposity, or diabetes.

All citations herein are incorporated herein by reference to the same extent as if each individual publication, patent application, or patent was specifically and individually indicated to be incorporated by reference including all figures and drawings.

Many modifications and variations of this invention, as will be apparent to one of ordinary skill in the art can be made to adapt to a particular situation, material, composition of matter, process, process step or steps, to preserve the objective, spirit and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of modulating an activity of a cell comprising contacting the cell with an agonist or antagonist of:
a) IL-19 (SEQ ID NOs:1 or 2); or
b) IL-24 (SEQ ID NOs:3 or 4);
wherein the cell modulates airway hyperreactivity or airway inflammation.

2. The method of claim 1, wherein the cell is:
a) a monocyte or macrophage;
b) a T cell or B cell;
c) a dendritic cell; or
d) an epithelial or endothelial cell.

3. The method of claim 2, wherein the macrophage is an alveolar macrophage.

4. The method of claim 1, wherein the agonist or antagonist is a binding composition derived from an antigen binding site of an antibody.

5. The method of claim 1, wherein the agonist or antagonist specifically binds to a polypeptide or nucleic acid of:
a) IL-19 (SEQ ID NOs:1 or 2);
b) IL-24 (SEQ ID NOs:3 or 4);
c) IL20R1;
d) IL-20R2; or
e) IL-22R.

6. The method of claim 5, wherein the agonist or antagonist comprises:
a) a polyclonal antibody;
b) a monoclonal antibody;
c) a humanized antibody;
d) an Fab, Fv, or F(ab′)₂ fragment;
e) a peptide mimetic of an antibody;
f) a nucleic acid; or
g) a detectable label.

7. A method of treating a subject suffering from an airway hyperreactivity disorder or an airway inflammatory disorder comprising administering an effective amount of an agonist or antagonist of:
a) IL-19 (SEQ ID NOs:1 or 2); or
b) IL-24 (SEQ ID NOs:3 or 4).

8. The method of claim 7, wherein the disorder is mediated by:
a) monocytes or macrophages;
b) T cells or B cells;
c) dendritic cells; or
d) epithelial or endothelial cells.

9. The method of claim 8, wherein the macrophages are alveolar macrophages.

10. The method of claim 7, wherein the disorder comprises:
a) asthma;
b) allergic rhinitis;
c) bronchitis;
d) bronchiolitis; or
e) chronic obstructive pulmonary disorder (COPD).

11. The method of claim 7, wherein the antagonist reduces or inhibits airway hyperreactivity.

12. The method of claim 7, wherein the agonist or antagonist is a binding composition derived from an antigen binding site of an antibody.

13. The method of claim 7, wherein the agonist or antagonist specifically binds to a polypeptide or nucleic acid comprising:
a) IL-19 (SEQ ID NO:1 or 2);
b) IL-24 (SEQ ID NO:3 or 4);
c) IL-20R1;
d) IL-20R2; or
e) IL-22R.

14. The method of claim 13, wherein the agonist or antagonist comprises:
a) a polyclonal antibody;
b) a monoclonal antibody;
c) a humanized antibody;
d) an Fab, Fv, or F(ab′)₂ fragment;
e) a peptide mimetic of an antibody;
f) a nucleic acid; or
g) a detectable label.

15. A method of diagnosing an airway hyperreactivity disorder or an airway inflammatory disorder comprising contacting a sample from a test subject with a binding composition that specifically binds to a polypeptide or nucleic acid of:
a) IL-19 (SEQ ID NO:1 or 2);
b) IL-24 (SEQ ID NO:3 or 4);
c) IL-20R1;
d) IL-20R2; or
e) IL-22R.

16. The method of claim 15, further comprising:
a) contacting the binding composition to a sample derived from a control subject or control sample; and
b) comparing the binding found with the test subject with the binding found with the control subject or control sample.

17. A method of screening for a compound that modulates a physiological activity, comprising:
a) contacting a candidate compound to an IL-19 knockout (IL-19KO) mouse to provide a contacted IL-19KO mouse, and determining the physiological activity in the contacted IL-19KO mouse;
b) determining the physiological activity in an IL-19KO mouse not contacted with the candidate compound; and
c) comparing the physiological activities of the contacted IL-19KO mouse and the non-contacted IL-19KO mouse.

18. The method of claim 17, wherein the physiological activity comprises:
a) an immune activity;
b) inflammation of the airway;
c) airway hyperreactivity; or
d) a proliferative activity.

19. A method of screening for a compound that modulates a physiological activity, comprising:
a) contacting a candidate compound to an IL-24 knockout (IL-24KO) mouse to provide a contacted IL-24KO mouse, and determining the physiological activity in the contacted IL-24KO mouse;
b) determining the physiological activity in an IL-24KO mouse not contacted with the candidate compound; and
c) comparing the physiological activities of the contacted IL-24KO mouse and the non-contacted IL-24KO mouse.

20. The method of claim 19, wherein the physiological activity comprises:
a) an immune activity;
b) inflammation of the airway;
c) airway hyperreactivity; or
d) a proliferative activity.

21. A method for treating or diagnosing obesity or diabetes comprising administering an effective amount of an agonist or antagonist of:
a) IL-19; or
b) IL-24.


SEQUENCE LISTING (8 sequences)

SEQ ID NO: 1 - DNA, 942 bp, Homo sapiens
  CDS (44)..(574)
ggcacgagcacaaggagcagcccgcaagcaccaagtgagaggaatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgtcctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtgaagtcatatagtccatgtctgtgatgtgagccaagtgatatcctgtagtacacattgtactgagtggtttttctgaataaattccatattttacctatggaaaaaaa

SEQ ID NO: 2 - protein, 177 aa, Homo sapiens [sequence omitted]

SEQ ID NO: 3 - DNA, 1700 bp, Homo sapiens
  CDS (356)..(892)
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 4 - protein, 179 aa, Homo sapiens
MQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 5 - DNA, 19 bp, Artificial
  Primer one for 372 base pair probe.
aagtgatttcgtttggctg

SEQ ID NO: 6 - DNA, 19 bp, Artificial
  Primer two for 372 base pair probe.
tgtctggtaagatcctatc

SEQ ID NO: 7 - DNA, 19 bp, Artificial
  Primer one for 322 base pair probe.
caccatcagcagcttggtc

SEQ ID NO: 8 - DNA, 19 bp, Artificial
  Primer two for 322 base pair probe.
taatcctcatgcagctctg